(19) United States
(12) Patent Application Publication
(10) Pub. No.: US 2002/0010334 A1
(43) Pub. Date: Jan. 24, 2002

(54) Processes to prepare pyrimidinediones

(76) Inventor: Xun Li, New Hope, PA (US)

Correspondence Address: Patent Administrator, FMC Corporation, 1735 Market Street, Philadelphia, PA, 19103, US

(21) Appl. No.: 09/887,972

(22) Filed: Jun. 22, 2001

Related U.S. Application Data
(60) Provisional application No. 60/215,228, filed on Jun. 30, 2000.

(51) Int. Cl. C07D 239/46

(52) U.S. Cl. 544/309

(57) ABSTRACT

A process for the preparation of a compound of formula I

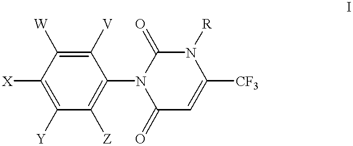

comprising the steps of forming an 1-unsubstituted pyrimidinedione by reacting under basic conditions either a carbamate or an isocyanate with an alkenoate and forming a compound of formula I by alkylating, aminating, haloalkylating, alkylnitrilating, arylating, allylating, alkylalkoxylating, alkylcarboxylating, or propargylating the 1-position of said unsubstituted pyrimidinedione by adding an adduct forming agent selected from the group consisting of alkylating, aminating, haloalkylating, alkylnitrilating, arylating, allylating, alkylalkoxylating, alkylcarboxylating, and propargylating agents; where the 1-unsubstituted pyrimidinedione, the carbamate, the isocyanate, the alkenoate, and substituents V, W, X, Y, Z, and R are described herein. The reactions as described herein are carried out in a single reaction vessel and often produce the pyrimidindione of formula I in near quantitative yields. It is emphasized that his abstract is provided to comply with the rules requiring an abstract that will allow a searcher or other reader to quickly ascertain the subject matter of the technical disclosure. It is submitted with the understanding that it will not be used to interpret or limit the scope or meaning of the claims (see 37 C.F.R. 1.72(b)).

This application claims benefit of U.S. Provisional Application No. 60/215,228, filed Jun. 30, 2000.

### FIELD OF THE INVENTION

The present invention relates generally to processes for the preparation of pyrimidinediones. In particular, it pertains to single vessel processes for preparing 1-substituted-3-(substituted phenyl)-pyrimidinediones from the appropriate carbamates or isocyanates.

### BACKGROUND OF THE INVENTION

It is known in the art that 1-substituted-3-(substituted phenyl)-pyrmidinediones are useful in the preparation of certain pesticides. For example, the use of 1-methyl-6-trifluoromethyl-3-(4-methoxyphenyl)-2,4(1H,3H)-pyrimidinedione as an intermediate to prepare herbicides is disclosed in U.S. Pat. Nos. 5,344,812, 5,399,543, 5,674,810 (issued to FMC Corportation on Sep. 6, 1994, Mar. 21, 1995, and Oct. 7, 1997, respectively); the use of 1-methyl-6-trifluoromethyl-3-(4-chloro-6-fluoro-3-methoxy-2-nitrophenyl)-2,4(1H,3H)-pyrimidinedione as an intermediate to prepare herbicides is disclosed in WO patent application 99/21837 (published on May 6, 1999 in the name of ISK Americas Incorporated); and the use of 1-methyl-6-trifluoromethyl-3-(4-chloro-2,6-difluorophenyl)-2,4(1H,3H)-pyrimidinedione as an intermediate to prepare herbicides is disclosed in WO patent application 00/28822 (published on May 25, 2000 in the name of BASF Aktiengesellschaft). In U.S. Pat. Nos. 5,344,812, 5,399,543, 5,674,810, the 1-methyl-6-trifluoromethyl-3-(4-methoxyphenyl)-2,4(1H,3H)-pyrimidinedione is prepared by reacting the corresponding phenylisocyanate with 3-amino-4,4,4-trifluorocrotonate, sodium hydride, and methyl iodide. Similar to the processes set forth in U.S. Pat. Nos. 5,344,812, 5,399,543, 5,674,810, in WO patent application 99/21837, the 1-methyl-6-trifluoromethyl-3-(4-chloro-6-fluoro-3-methoxy-2-nitrophenyl)-2,4(1H,3H)-pyrimidinedione is prepared by reacting the corresponding phenylisocyanate with ethyl-3-amino-4,4,4-trifluorocrotonate, sodium hydride, and methyl iodide. Similar to the processes set forth in U.S. Pat. Nos. 5,344,812, 5,399,543, 5,674,810 and WO patent application 99/21837, in WO patent application 00/28822, the 1-methyl-6-trifluoromethyl-3-(4-chloro-2,6-difluorophenyl)-2,4(1H,3H)-pyrimidinedione is prepared by reacting the corresponding phenylisocyanate with ethyl-3-amino-4,4,4-trifluorocrotonate and sodium hydride to form 6-trifluoromethyl-3-(4-chloro-2,6-difluorophenyl)-2,4(1H,3H)-pyrimidinedione that, in turn, is reacted with methyl iodide to form the 1-methyl-6-trifluoromethyl-3-(4-chloro-2,6-difluorophenyl)-2,4(1H,3H)-pyrimidinedione. These processes result in low yields of product and as such are not commercially viable. Accordingly, there exists a need for further processes for preparing 1-substituted-3-(substituted phenyl)-pyrimidinediones.

### SUMMARY OF THE INVENTION

The present invention describes processes for preparing pyrimidinediones from carbamates or isocyanates, often in near quantitative yields.

One aspect of the present invention involves processes of preparing pyrimidinediones of formula I:

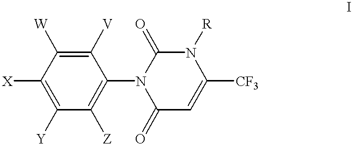

wherein V, W, X, Y, and Z are each independently selected from hydrogen, halogen, cyano, nitro, alkyl, alkoxyalkyl, phenylalkyl, alkenyl, alkenyloxyalkyl, alkynyl, alkynyloxyalkyl, haloalkyl, haloalkenyl, haloalkynyl, alkylthioalkyl, alkenylthioalkyl, alkynylthioalkyl, alkylsulfinylalkyl, alkenylsulfinylalkyl, alkynylsulfinylalkyl, alkylsulfonylalkyl, alkenylsulfonylalkyl, alkynylsulfonylalkyl, phenoxyalkyl, phenylthioalkyl, phenylsulfinylalkyl, phenylsulfonylalkyl, hydroxy, alkoxy, cyanoalkoxy, alkoxyalkoxy, alkenyloxy, alkynyloxy, haloalkoxy, haloalkenyloxy, haloalkynyloxy, alkoxycarbonylalkoxy, amino, dialkylamino, dialkoxyamino, carboxy, alkoxycarbonyl, alkylthiocarbonyl, alkoxyalkoxycarbonyl, aminoacarbonyl, dialkylaminocarbonyl, and dialkoxyaminocarbonyl, wherein phenyl is optionally substituted with halogen, alkyl, or haloalkyl; and

R is selected from the group consisting of alkyl, amino, haloalkyl, alkylnitrilyl, aryl, allyl, alkylalkoxy, alkylcarboxyl, or propargyl;

by reacting under basic conditions a carbamate of formula B:

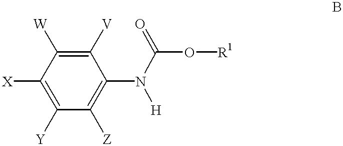

wherein R¹ is selected from the group consisting of hydrogen, alkyl, haloalkyl, aryloxy, substituted or unsubstituted aryl, substituted or unsubstituted heterocyclyl, the substituents of said substituted aryl or heterocyclyl comprising one or more members selected from the group consisting of halo, C₁₋₂₀ alkyl or alkoxy, nitro, amino, amido, alkylthio, aryl, arylthio, aryloxy, alkylsulfonyl, and arylsulfonyl;

with an alkenoate of formula D:

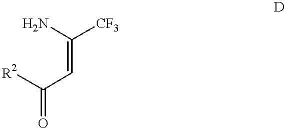

wherein R² is alkoxy;

to form a 1-unsubstituted pyrimidinedione of formula A:

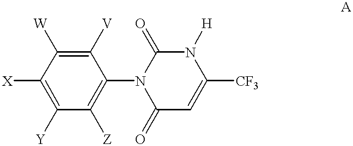

and reacting the unsubstituted pyrimidinedione A with an adduct forming agent selected from the group consisting of alkylating, aminating, haloalkylating, alkylnitrilating, arylating, allylating, alkylalkoxylating, alkylcarboxylating, and propargylatings agents to form the pyrmidinedione of formula I.

Another aspect of the present invention involves processes of preparing the pyrmidinediones of formula I by reacting under basic conditions an isocyanate of formula C:

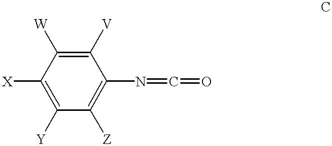

with the alkenoate of formula D to form the 1-unsubstituted pyrimidinedione of formula A and reacting the unsubstituted pyrimidinedione A with the adduct forming agent to form the pyrmidinedione of formula I.

Also provided in accordance with the present invention is a process for preparing 1-methyl-6-trifluoromethyl-3-(4-methoxyphenyl)-2,4(1H,3H)-pyrimidinedione by reacting ethyl 4-methoxyphenylcarbamate with ethyl 3-amino-4,4,4-trifluoro-2-butenoate under basic conditions to form 6trifluoromethyl-3-(4-methoxyphenyl)-2,4(1H,3H)-pyrimidinedione and then reacting the 6-trifluoromethyl-3-(4-methoxyphenyl)-2,4(1H,3H)-pyrimidinedione with a methyl halide to form the 1-methyl-6-trifluoromethyl-3-(4-methoxyphenyl)-2,4(1H,3H)-pyrimidinedione.

### DETAILED DESCRIPTION OF THE INVENTION

In one aspect of the present invention, a process is provided for preparing a pyrimidinedione of formula I, preferably in near quantitative yields. The process comprises the steps of:

forming a 1-unsubstituted pyrimidinedione of formula A:

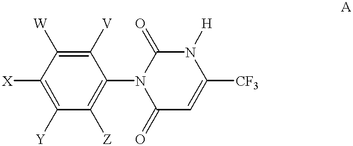

by reacting under basic conditions a carbamate of formula B:

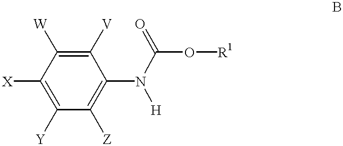

wherein R¹ is as defined above;

with an alkenoate of formula D:

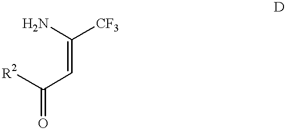

wherein R² is as defined above; and

forming a compound of formula I by reacting said unsubstituted pyrimidinedione A with an adduct forming agent selected from the group consisting of alkylating, aminating, haloalkylating, alkylnitrilating, arylating, allylating, alkylalkoxylating, alkylcarboxylating, and propargylating agents;

wherein said reactions are carried out within a single reaction vessel.

Preferred processes are those in which V, W, X, Y, and Z are each independently selected from hydrogen, halogen, alkyl, nitro, haloalkyl, and alkoxy; R is alkyl or amino; R¹ is alkyl; and R² is ethoxy.

Particularly preferred processes are those in which V, W, Y and Z are hydrogen; X is alkoxy; R is alkyl; and R¹ is ethyl. An even more preferred process is that in which X is methoxy and R is methyl.

Suitable bases that may used are those substances that have the ability to react with an acid to form a salt without hydrolyzing the alkenoate D. Examples of bases that can be used include, but are not limited to, sodium hydride, sodium methoxide, potassium methoxide, potassium carbonate, sodium carbonate, cesium carbonate, lithium carbonate, and ammonium carbonate. Preferred bases that can be used include sodium hydride, sodium methoxide, and potassium carbonate. A particularly preferred base is potassium carbonate.

Suitable alkylating, aminating, haloalkylating, alkylnitrilating, arylating, allylating, alkylalkoxylating, alkylcarboxylating, or propargylating agents that can be used as adduct forming agents are those agents that have the ability to attach an alkyl, amino, haloalkyl, alkylnitrilyl, aryl, allyl, alkylalkoxy, alkylcarboxyl, or propargyl moiety, respectively, at the 1-position of the unsubstituted pyrimdinedione A. Examples of such agents that can be used include, but are not limited to, methyl iodide, methyl chloride, methyl bromide, 1-aminooxysulfonyl-2,4,6-trimethylbenzene, O-(2,4-dinitrophenyl)hydroxylamine, hydroxylamine-O-sulfonic acid, allyl bromide, propargyl bromide, methoxymethyl bromide, benzyl chloride, and ethyl chloroacetate. Preferred agents that can used are methyl iodide, methyl bromide, 1-aminooxysulfonyl-2,4,6-trimethylbenzene, and hydroxylamine-O-sulfonic acid.

In the present invention, the reaction of the carbamate B with the alkenoate D to form the unsubstituted pyrimidinedione A is preferably carried out at elevated temperature, such as from about 70° C. to about 170° C., more preferably from about 100° C. to about 160° C., preferably for about three to about 24 hours, more preferably for about five to about 18 hours. The reaction can be run at lower temperatures, but generally will require an appreciably longer time to complete. In addition, the reaction may be run at atmospheric or increased pressure.

One molar equivalent of alkenoate D can be reacted with about 0.01 to about 5 molar equivalents, preferably about 0.5 to about 3 molar equivalents, of carbamate B and about 0.5 to about 10 molar equivalents, preferably about 1 to about 5 molar equivalents, of base.

The reaction of the carbamate B with the alkenoate D to form the unsubstituted pyrimidinedione A can be carried out neat or in a suitable organic solvent. Preferred organic solvents, both polar and apolar, useful in the context of the present invention include halogenated solvents, for example, without limitation, chlorobenzene, carbon tetrachloride, bromodichloromethane, dibromochloromethane, bromoform, chloroform, bromochloromethane, butyl chloride, dichloromethane, tetrachloroethylene, trichloroethylene, 1,1,1-trichloroethane, 1,1,2-trichloroethane, 1,1-dichloroethane, 2-chloropropane, hexafluorobenzene, 1,2,4-trichlorobenzene, 1,2-dichlorobenzene, fluorobenzene and other halogenated solvents known in the art.

Preferred polar organic solvents include ethers, for example, without limitation, dimethoxymethane, tetrahydrofuran (THF), 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, tert.-butyl ethyl ether, tert.-butyl methyl ether and other ether solvents known in the art. Other polar organic solvents useful in the context of the present invention include, for example, without limitation, propionitrile, ethyl formate, methyl acetate, hexachloroacetone, acetone, ethyl methyl ketone, ethyl acetate, nitromethane, nitrobenzene, glymes, and other polar solvents known in the art.

Other organic solvents useful herein include polar aprotic solvents, for example, without limitation, N,N-dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, sulfolane, N,N-dimethylpropionamide, tetramethylurea, hexamethylphosphoramide and other polar aprotic solvents known in the art.

Yet other organic solvents useful for implementation of the present invention include protic solvents, for example, without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, isobutanol, tert.-butanol, 2-ethoxyethanol, diethylene glycol, 1-, 2-, or 3-pentanol, 2,2-dimethyl-1-propanol, tert.-pentanol, cyclohexanol, anisole, benzyl alcohol, glycerol and other protic solvents known in the art.

Further organic solvents useful in the context of the present invention include: acidic solvents, for example, without limitation, trifluoroacetic acid, acetic acid, formic acid and other acidic solvents known in the art; basic solvents, for example, without limitation, 2-, 3-, or 4-picoline, pyrrole, pyrrolidine, morpholine, pyridine, piperidine, triethylamine and other basic solvents known in the art; and hydrocarbon solvents, for example, without limitation, benzene, cyclohexane, pentane, hexane, toluene, cycloheptane, methylcyclohexane, heptane, ethylbenzene, ortho-, meta-, or para-xylene, octane, indane, nonane, naphthaline and other hydrocarbon solvents known in the art.

Organic solvents particularly suitable for forming the unsubstituted pyrimidinedione A are those that are low in cost, enhance the solubility of the starting materials to promote rate of reaction, and offer minimum solvent decomposition. Preferred organic solvents include DMF, DMAC, DMPU, DMI, NMP, formamide, N-methylacetamide, N-methylformamide, acetonitrile, dimethyl sulfoxide, sulfolane, N,N-dimethylpropionamide, tetramethylurea, hexamethylphosphoramide. More preferred solvents include DMF, DMAC, acetonitrile, and dimethyl sulfoxide. A particularly preferred organic solvent in which to conduct the formation of the unsubstituted pyrimidinedione A is DMF.

In the course of conducting chemical reactions, especially large scale organic chemical reactions yielding commercial quantities of desired product, a balance must be met between having to handle too much solvent yet providing sufficient solvent to afford optimum reaction conditions. A useful ratio of solvent to alkenoate D to afford optimum reaction conditions is generally in the range of about 2.5/1 to about 40/1 wt/wt, preferably about 3/1 to about 20/1.

The reaction of the unsubstituted pyrimidinedione A with the adduct forming agent to form the pyrimidinedione of formula I is generally carried out at about 0° C. to about 80° C., preferably about 0° C. to about 40° C., for at least 30 minutes, preferably for about one to about eight hours. Similar to above, the reaction of unsubstituted pyrimidinedione A with the adduct forming agent can be carried out at atmospheric or increased pressure.

The reaction of unsubstituted pyrimidinedione A with the adduct forming agent can be carried out by combining one molar equivalent of the unsubstituted pyrimidinedione A with about 0.01 to about 10 molar equivalents, preferably about 1 to about 4 molar equivalents, of the adduct forming agent.

Similar to the reaction of the carbamate B with alkenoate D to form the unsubstituted pyrimidinedione A, the reaction of unsubstituted pyrimidinedione A with the adduct forming agent can be carried out neat or in a suitable organic solvent. The organic solvents and ratios disclosed above can also be used in the reaction of the unsubstituted pyrimidinedione A with the adduct forming agent. Preferred organic solvents that may be used in the reaction of the unsubstituted pyrimidinedione A with the adduct forming agent are DMF, DMAC, acetonitrile, and dimethyl sulfoxide. The particularly preferred organic solvent in which to conduct the formation of the pyrimidinedione I is DMF.

In another aspect of the present invention, a process is provided for the preparing a pyrimidinedione of formula I, as set forth above and wherein V, W, X, Y, Z, R are as defined above with the proviso that X is not alkoxy, (preferably in near quantitative yields), which process comprising the steps of:

forming a 1-unsubstituted pyrimidinedione of formula A, as set forth above, by reacting under basic conditions an isocyanate of formula C:

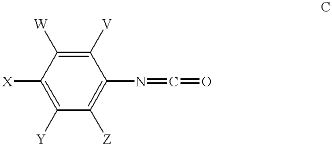

with an alkenoate of formula D, as set forth above and wherein R² is as defined above, and

forming a compound of formula I by reacting said unsubstituted pyrimidinedione A with an adduct forming agent selected from the group consisting of alkylating, aminating, haloalkylating, alkylnitrilating, arylating, allylating, alkylalkoxylating, alkylcarboxylating, and propargylating agents; wherein said reactions are carried out within a single reaction vessel.

The bases set forth above may also be used when the isocyanate C is reacted with the alkenoate D to form the unsubstituted pyrimdinedione A. Preferred bases that can be used in the reaction of the isocyanate C with the alkenoate D to form the unsubstituted pyrimdinedione A include sodium hydride, sodium methoxide, and potassium carbonate. A particularly preferred base is potassium carbonate.

The alkylating, aminating, haloalkylating, alkylnitrilating, arylating, allylating, alkylalkoxylating, alkylcarboxylating, or propargylating agents set forth above can be used as adduct forming agents in conjunction the isocyanate C. Preferred agents that can used in the conjunction with the isocyanate C are methyl iodide, methyl bromide, 1-aminooxysulfonyl-2,4,6-trimethylbenzene, and hydroxylamine-O-sulfonic acid.

The reaction of the isocyanate C with the alkenoate D to form the unsubstituted pyrimidinedione A is preferably carried out at elevated temperature, such as from about 70° C. to about 170° C., more preferably from about 100° C. to about 160° C., preferably for about three to about 24 hours, more preferably for about five to about 18 hours. The reaction can be run at lower temperatures, but generally will require an appreciably longer time to complete. In addition, the reaction may be run at atmospheric or increased pressure.

One molar equivalent of alkenoate D can be reacted with about 0.01 to about 5 molar equivalents, preferably about 0.5 to about 3 molar equivalents, of isocyanate C and about 0.5 to about 10 molar equivalents, preferably about 1 to about 5 molar equivalents, of base.

The reaction of the isocyante C with the alkenoate D to form the unsubstituted pyrimidinedione A can be carried out neat or in a suitable organic solvent. The organic solvents and ratios set forth above, including, but not limited to, the preferred solvents and ratios, can also be used in carrying out the reaction of the isocyante C with the alkenoate D to form the unsubstituted pyrimidinedione A.

The unsubstituted pyrimidinedione A can be reacted with the adduct forming agent at about 0° C. to about 80° C., preferably about 0° C. to about 40° C., for at least 30 minutes, preferably for about one to about eight hours, to form the pyrimidinedione of formula I. The reaction of unsubstituted pyrimidinedione A with the adduct forming agent can be carried out at atmospheric or increased pressure.

The reaction of unsubstituted pyrimidinedione A with the adduct forming agent can be carried out by combining one molar equivalent of the unsubstituted pyrimidinedione A with about 0.01 to about 10 molar equivalents, preferably about 1 to about 4 molar equivalents, of the adduct forming agent.

The reaction of the unsubstituted pyrimidinedione A with the adduct forming can be carried out neat or in a suitable organic solvent. The organic solvents and ratios disclosed above can also be used in conjunction with the reaction of unsubstituted pyrimidinedione A with the adduct forming agent. Preferred organic solvents that may be used in conjunction with the reaction of unsubstituted pyrimidinedione A with the adduct forming agent are DMF, DMAC, acetonitrile, and dimethyl sulfoxide. The particularly preferred organic solvent in which to conduct the formation of the pyrimidinedione I is DMF.

In yet another aspect of the present invention, 1-methyl-6-trifluoromethyl-3-(4-methoxyphenyl)-2,4(1H,3H)-pyrimidinedione can be prepared (preferably in near quantitative yields in a single reaction vessel) by reacting ethyl 4-methoxyphenylcarbamate with ethyl 3-amino-4,4,4-trifluoro-2-butenoate under basic conditions to form 6-trifluoromethyl-3-(4-methoxyphenyl)-2,4(1H,3H)-pyrimidinedione and then methylating the 6-trifluoromethyl-3-(4-methoxyphenyl)-2,4(1H,3H)-pyrimidinedione in the 1-position with a methyl halide, such as methyl iodide or bromide, to form the 1-methyl-6-trifluoromethyl-3-(4-methoxyphenyl)-2,4(1H,3H)-pyrimidinedione.

As used in this specification and unless otherwise indicated the substituent terms “alkyl”, “cycloalkyl”, “alkoxy”, “aryloxy”, and “alkoxyarylamino”, used alone or as part of a larger moiety, include straight or branched chains of at least one or two carbon atoms, as appropriate to the substituent, and preferably up to 20 carbon atoms, more preferably up to ten carbon atoms, even more preferably up to seven carbon atoms. “Halogen” or “halo” refers to fluorine, bromine, iodine, or chlorine. “Aryl” refers to an aromatic ring structure having 5 to 10 carbon atoms. “Heteroaryl” refers to an aromatic ring structure having 1 to 4 nitrogen, sulfur, or oxygen atoms or a combination thereof as hetero ring components, with the balance being carbon atoms. The term “ambient temperature” as utilized herein shall mean any suitable temperature found in a laboratory or other working quarter, and is generally not below about 15° C. nor above about 30° C.

The processes of the present invention are typically safer and more efficient than existing methods to the extent they reduce the risk of chemical exposure resulting from the transfer of chemicals from one reaction vessel to another, the reagents used are cheaper, and the time of the reaction is reduced. In addition to these advantages, the processes of the present invention generally convert in excess of 70%, often in excess of 90%, of the starting material to the pyrimidinedione I.

The present invention is now described in more detail by reference to the following examples, but it should be understood that the invention is not construed as being limited thereto.

### EXAMPLE 1

This example illustrates one protocol for the preparation of 1-methyl-6-trifluoromethyl-3-(4-methoxyphenyl)-2,4(1H,3H)-pyrimidinedione using methyl iodide as the alkylating agent.

To a 100 mL roundbottom flask equipped with a mechanical stirrer and thermometer was added 23.1 grams (0.118 mole-0.94 equiv.) of 99% pure ethyl 4-methoxyphenylcarbamate, 23 grams (0.125 mole-1 equiv.) of ethyl 3-amino-4,4,4-trifluoro-2-butenoate (available from Aldrich Chemical Company, Milwaukee, Wis.), 21.9 grams (0.159 mole-1.3 equiv.) of 98% pure potassium carbonate (available from Aldrich Chemical Company), and 125 mL (% wt/wt. butenoate to solvent-18.4%) of DMF (available from J. T. Baker Inc., Phillipsburg, N.J.). The reaction mixture was heated at reflux for six hours. After this time, the reaction mixture was allowed to cool to 38° C. and then 19.7 grams (0.138 moles-1.1 equiv.) of methyl iodide (available from Aldrich Chemical Company) was added dropwise during a 15 minute period. Upon completion of addition, the reaction mixture was heated to 80° C. and stirred at that temperature for two hours. At the conclusion of this period, the DMF was removed under reduced pressure at 100° C. and 125 mL of water was added. Upon completion of addition, the resulting mixture was allowed to cool to ambient temperature during a one hour period. The resulting precipitate was collected by filtration and dried in an oven at 60° C. for four hours, yielding 32.2 grams (88.4%) of 97.3% pure 1-methyl-6-trifluoromethyl-3-(4-methoxyphenyl)-2,4(1H,3H)-pyrimidinedione. The NMR spectrum was consistent with the proposed structure.

### EXAMPLE 2

This example illustrates one protocol for the preparation of 1-methyl-6-trifluoromethyl-3-(4-methoxyphenyl)-2,4(1H,3H)-pyrimidinedione using methyl bromide as the alkylating agent.

To a two liter three-necked flask equipped with a thermocouple, Dean-Stark trap, nitrogen inlet, a mechanical stirrer and a thermometer was added one liter of DMF, 200 grams (1.0 mole-0.91 equiv.) of 96% pure ethyl 4-methoxyphenylcarbamate, 199 grams (1.1 moles-1 equiv.) of 97% pure ethyl 3-amino-4,4,4-trifluorobutenoate (% wt/wt. butenoate to solvetnt-19.9%) and 190 grams (1.4 moles-1.3 equiv.) of potassium carbonate. The reaction mixture was heated to reflux where it stirred for eight hours. After this time, the DMF was removed by distillation and the reaction mixture was analyzed by Gas Chromatography (GC), which indicated the formation of the 1-unsubstituted pyrimidinedione. The reaction mixture was cooled to ambient temperature and a solution of 107 grams (1.13 moles-1.02 equiv.) of methyl bromide (available from Aldrich Chemical Company) and 190 grams (1.4 moles-1.3 equiv.) of potassium carbonate in one liter (% wt/wt. butenoate to solvent-19.9%) of DMF was added. Upon completion of addition, the reaction mixture was stirred at ambient temperature for one hour and the resulting precipitate was collected by filtration. The filter cake was washed with two 100 mL portions of distilled water, air-dried for two hours, and then concentrated under reduced pressure, yielding 286 grams (91% yield) of 97% pure 1-methyl-6-trifluoromethyl-3-(4-methoxyphenyl)-2,4(1H,3H)-pyrimidinedione. The NMR spectrum was consistent with the proposed structure.

### EXAMPLE 3

This example illustrates one protocol for the preparation of l-amino-6-trifluoromethyl-3-(4-methoxyphenyl)-2,4(1H,3H)-pyrimidinedione using hydroxylamine-O-sulfonic acid as the aminating agent.

To a 100 mL roundbottom flask equipped with a mechanical stirrer and thermometer is added 23.1 grams (0.118 mole-0.94 equiv.) of ethyl 4-methoxyphenylcarbamate, 23 grams (0.125 mole-1 equiv.) of ethyl 3-amino-4,4,4-trifluoro-2-butenoate, 21.9 grams (0.159 mole-1.3 equiv.) of potassium carbonate, and 125 mL (% wt/wt. butenoate to solvent-18.4%) of DMF. The reaction mixture is heated at reflux for eight hours. After this time, the reaction mixture is cooled to ambient temperature and then 15.6 grams (0.138 moles-1.1 equiv.) of hydroxylamine-O-sulfonic acid (available from Aldrich Chemical Company) is added. Upon completion of addition, the reaction mixture is heated to 80° C. where it is stirred for two hours. At the conclusion of this period, the DMF is removed under reduced pressure and 125 mL of water is added. Upon completion of addition, the resulting mixture is allowed to cool to ambient temperature. The resulting precipitate is collected by filtration and dried to yield 1-amino-6-trifluoromethyl-3-(4-methoxyphenyl)-2,4(1H,3H)-pyrimidinedione.

### EXAMPLE 4

This example illustrates one protocol for the preparation of 1-methyl-6-trifluoromethyl-3-(4-chloro-2-fluorophenyl)-2,4(1H,3H)-pyrimidinedione using methyl iodide as the alkylating agent.

To a 500 mL roundbottom flask equipped with a thermometer, condenser, and an overhead stirrer was added 18.9 grams (0.1 mole-1.0 equiv.) of 97% pure ethyl 3-amino-4,4,4-trifluoro-2-butenoate, 13.8 grams (0.1 mole-1.0 equiv.) of potassium carbonate, and 100 mL (% wt/wt. butenoate to solvent-18.9%) of DMF. The reaction mixture was heated to reflux and a solution of 24.6 grams (0.11 mole-1.1 equiv.) of 97% pure ethyl (4-chloro-2-fluorophenyl)carbamate in 50 mL of DMF (% wt/wt. butenoate to solvent-37.8%) was added dropwise. Upon completion of addition, the reaction mixture was heated at reflux for 7.5 hours. After this time, the reaction mixture was cooled to ambient temperature where it was allowed to stand for about 18 hours. At the conclusion of this period, 21.4 grams (0.151 mole-1.5 equiv.) of methyl iodide was added dropwise. Upon completion of addition, the reaction mixture was heated to 95° C. where it stirred for 24 hours. After this time, the reaction mixture was again cooled to ambient temperature and the mixture was concentrated under reduced pressure to yield a gummy brown solid. To the brown solid was added 100-110 mL of water. The resulting mixture was stirred for about one hour. The resulting precipitate was collected by filtration, placed in a crystallizing dish, and dried at 60° C. for about 18 hours. The resulting solid was washed with diethyl ether and then purified by chromatographic techniques to yield 1-methyl-6-trifluoromethyl-3-(4-chloro-2-fluorophenyl)-2,4(1H,3H)-pyrimidinedione.

### EXAMPLE 5

This example illustrates one protocol for the preparation of 1-methyl-6-trifluoromethyl-3-(4-methoxyphenyl)-2,4(1H,3H)-pyrimidinedione using 4-methoxyphenylisocyanate as the starting material.

A stirred mixture of 3.0 grams (0.02 mole-1.0 equiv.) of 99% pure ethyl 4-methoxyphenylisocyanate (available from Aldrich Chemical Company), 3.8 grams (0.02 mole-1.0 equiv.) of 97% pure ethyl 3-amino-4,4,4-trifluoro-2-butenoate, 2.8 grams (0.02 mole-1.0 equiv.) of potassium carbonate, and 20 mL (% wt/wt. butenoate to solvent-19%) of DMF was heated to 140° C. for one hour. At the conclusion of this period, the reaction mixture was analyzed by GC, which indicated the reaction was complete. The reaction mixture was allowed to cool to ambient temperature and 6.6 grams (0.047 mole-2.4 equiv.) of 99.5% pure methyl iodide was added. Upon completion of addition, the reaction mixture was heated to 80° C. where it stirred for one hour. After this time, the reaction mixture was allowed to cool to ambient temperature. Once at the prescribed temperature, the reaction mixture was concentrated under reduced pressure to yield a residue. To the residue was added 40 mL of water followed by 80 mL of ethyl acetate. The pH of the resulting solution was adjusted to a pH of 7 with concentrated hydrochloric acid. The organic layer was separated and concentrated under reduced pressure to yield 4.7 grams of a solid. The solid was concentrated under reduced pressure to yield 4.2 grams (70% yield) of 94.3% pure 1-methyl-6-trifluoromethyl-3-(4-methoxyphenyl)-2,4(1H,3H)-pyrimidinedione. The NMR spectrum was consistent with the proposed structure.

While the invention has been described in detail and with reference to specific embodiments thereof, it will be apparent to one skilled in the art that various changes and modifications can be made therein without departing from the spirit and scope thereof.

I claim:

1. A process for the preparation of a compound of formula I: 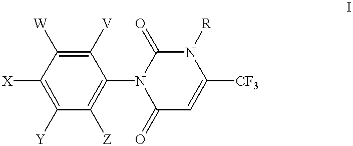
wherein V, W, X, Y, and Z are each independently selected from hydrogen, halogen, cyano, nitro, alkyl, alkoxyalkyl, phenylalkyl, alkenyl, alkenyloxyalkyl, alkynyl, alkynyloxyalkyl, haloalkyl, haloalkenyl, haloalkynyl, alkylthioalkyl, alkenylthioalkyl, alkynylthioalkyl, alkylsulfinylalkyl, alkenylsulfinylalkyl, alkynylsulfinylalkyl, alkylsulfonylalkyl, alkenylsulfonylalkyl, alkynylsulfonylalkyl, phenoxyalkyl, phenylthioalkyl, phenylsulfinylalkyl, phenylsulfonylalkyl, hydroxy, alkoxy, cyanoalkoxy, alkoxyalkoxy, alkenyloxy, alkynyloxy, haloalkoxy, haloalkenyloxy, haloalkynyloxy, alkoxycarbonylalkoxy, amino, dialkylamino, dialkoxyamino, carboxy, alkoxycarbonyl, alkylthiocarbonyl, alkoxyalkoxycarbonyl, aminoacarbonyl, dialkylarninocarbonyl, and dialkoxyaminocarbonyl, wherein phenyl is optionally substituted with halogen, alkyl, or haloalkyl; and
R is selected from the group consisting of alkyl, amino, haloalkyl, alkylnitrilyl, aryl, allyl, alkylalkoxy, alkylcarboxyl, or propargyl;
comprising the steps of:
forming a 1-unsubstituted pyrimidinedione of formula A: 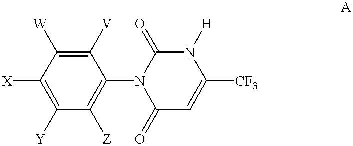
by reacting under basic conditions a carbamate of formula B: 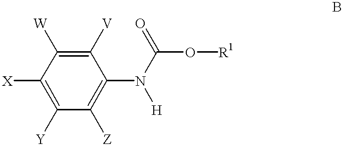
wherein R¹ is selected from the group consisting of hydrogen, alkyl, haloalkyl, aryloxy, substituted or unsubstituted aryl, substituted or unsubstituted heterocyclyl, the substituents of said substituted aryl or heterocyclyl comprising one or more members selected from the group consisting of halo, C₁₋₂₀ alkyl or alkoxy, nitro, amino, amido, alkylthio, aryl, arylthio, aryloxy, alkylsulfonyl, and arylsulfonyl;
with an alkenoate of formula D: 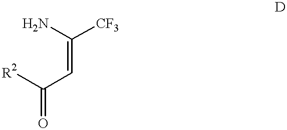
wherein R² is alkoxy;
and
forming a compound of formula I by reacting said unsubstituted pyrimidinedione A with an adduct forming agent selected from the group consisting of alkylating, aminating, haloalkylating, alkylnitrilating, arylating, allylating, alkylalkoxylating, alkylcarboxylating, and propargylating agents; wherein said reactions are carried out within a single reaction vessel.

2. The process of claim 1, wherein V, W, X, Y, and Z are each independently selected from hydrogen, halogen, alkyl, nitro, haloalkyl, and alkoxy; R is alkyl or amino; R¹ is alkyl; and R² is ethoxy.

3. The process of claim 2, wherein V, W, Y and Z are hydrogen; X is alkoxy; R is alkyl; and R¹ is ethyl.

4. The process of claim 3, wherein X is methoxy and R is methyl.

5. The process of claim 1, wherein the base is selected from the group consisting of sodium hydride, sodium methoxide, potassium methoxide, potassium carbonate, sodium carbonate, cesium carbonate, lithium carbonate, and ammonium carbonate.

6. The process of claim 1, wherein the base is selected from the group consisting of sodium hydride, sodium methoxide, and potassium carbonate.

7. The process of claim 1, wherein the adduct forming agent is selected from the group consisting of methyl iodide, methyl chloride, methyl bromide, 1-aminooxysulfonyl-2,4,6-trimethylbenzene, O-(2,4-dinitrophenyl)hydroxylamine, hydroxylamine-O-sulfonic acid, allyl bromide, propargyl bromide, methoxymethyl bromide, benzyl chloride, and ethyl chloroacetate.

8. The process of claim 1, wherein the steps of reacting the carbamate B with alkenoate D to form the unsubstituted pyrimidinedione A and reacting said unsubstituted pyrimidinedione A with the adduct forming agent are carried out in an organic solvent.

9. The process of claim 8, wherein the organic solvent is a polar aprotic solvent.

10. The process of claim 9, wherein the organic solvent is selected from the group consisting of N,N-dimethylformamide, dimethylacetamide, 1.3-dimethyl-3,4,5,6-tetrahydro-2(1H)-pyrimidinone, 1,3-dimethyl-2-imidazolidinone, N-methylpyrrolidinone, formamide, N-methylacetamide, N-methylformamide, acetonitrile, dimethyl sulfoxide, sulfolane, N,N-dimethylpropionamide, tetramethylurea, hexamethylphosphoramide

11. The process of claim 1, wherein the step of reacting the carbamate B with alkenoate D to form the unsubstituted pyrimidinedione A comprises heating the reaction mixture at about 70° C. to about 170° C. for about three to about 24 hours.

12. The process of claim 11, wherein the step of reacting the carbamate B with alkenoate D to form the unsubstituted pyrimidinedione A comprises heating the reaction mixture at about 100° C. to about 160° C. for about five to about 18 hours.

13. The process of claim 1, wherein the step of reacting the unsubstituted pyrimidinedione A with the adduct forming agent comprises reacting the unsubstituted pyrimidinedione A and adduct forming agent at about 0° C. to about 80° C. for at least 30 minutes.

14. The process of claim 13, wherein the step of reacting the unsubstituted pyrimidinedione A with the adduct forming agent comprises reacting the unsubstituted pyrimidinedione A and adduct forming agent at 0° C. to 40° C. for about one to about eight hours.

15. The process of claim 1, wherein:
about 0.01 to about 5 molar equivalents of carbamate B are present per one molar equivalent of alkenoate D;
about 0.5 to about 10 molar equivalents of base are present per one molar equivalent of alkenoate D; and
about 0.01 to about 10 molar equivalents of adduct forming agent are present per one molar equivalent of unsubstituted pyrimidinedione A.

16. The process of claim 15, wherein:
about 0.5 to about 3 molar equivalents of of carbamate B are present per one molar equivalent of alkenoate D;
about 1 to about 5 molar equivalents of base are present per one molar equivalent of alkenoate D; and
about 1 to about 4 molar equivalents of adduct forming agent are present per one molar equivalent of unsubstituted pyrimidinedione A.

17. A process for preparing 1-methyl-6-trifluoromethyl-3-(4-methoxyphenyl)-2,4(1H,3H)-pyrimidinedione comprising the steps of:
reacting ethyl 4-methoxyphenylcarbamate with ethyl 3-amino-4,4,4-trifluoro-2-butenoate under basic conditions to form 6-trifluoromethyl-3-(4-methoxyphenyl)-2,4(1H,3H)-pyrimidinedione; and
reacting said 6-trifluoromethyl-3-(4-methoxyphenyl)-2,4(1H,3H)-pyrimidinedione with a methyl halide to form said 1-methyl-6-trifluoromethyl-3-(4-methoxyphenyl)-2,4(1H,3H)-pyrimidinedione;
wherein said process is carried out in a single reaction vessel.

18. The process of claim 23, wherein the methyl halide is methyl iodide or methyl bromide.

19. A process for the preparation of a compound of formula I: 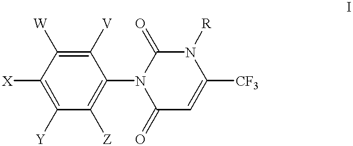
wherein V, W, X, Y, and Z are each independently selected from hydrogen, halogen, cyano, nitro, alkyl, alkoxyalkyl, phenylalkyl, alkenyl, alkenyloxyalkyl, alkynyl, alkynyloxyalkyl, haloalkyl, haloalkenyl, haloalkynyl, alkylthioalkyl, alkenylthioalkyl, alkynylthioalkyl, alkylsulfinylalkyl, alkenylsulfinylalkyl, alkynylsulfinylalkyl, alkylsulfonylalkyl, alkenylsulfonylalkyl, alkynylsulfonylalkyl, phenoxyalkyl, phenylthioalkyl, phenylsulfinylalkyl, phenylsulfonylalkyl, hydroxy, alkoxy, cyanoalkoxy, alkoxyalkoxy, alkenyloxy, alkynyloxy, haloalkoxy, haloalkenyloxy, haloalkynyloxy, alkoxycarbonylalkoxy, amino, dialkylamino, dialkoxyamino, carboxy, alkoxycarbonyl, alkylthiocarbonyl, alkoxyalkoxycarbonyl, aminoacarbonyl, dialkylarninocarbonyl, and dialkoxyaminocarbonyl, wherein phenyl is optionally substituted with halogen, alkyl, or haloalkyl, with the proviso that X is not alkoxy; and
R is selected from the group consisting of alkyl, amino, haloalkyl, alkylnitrilyl, aryl, allyl, alkylalkoxy, alkylcarboxyl, or propargyl;
comprising the steps of:
forming a 1-unsubstituted pyrimidinedione of formula A: 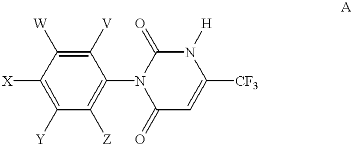
by reacting under basic conditions an isocyanate of formula C: 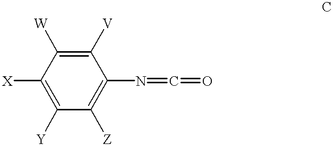
with an alkenoate of formula D: 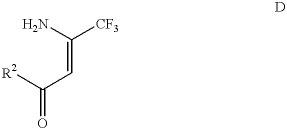
wherein R² is alkoxy;
and
forming a compound of formula I by reacting said unsubstituted pyrimidinedione A with an adduct forming agent selected from the group consisting of alkylating, aminating, haloalkylating, alkylnitrilating, arylating, allylating, alkylalkoxylating, alkylcarboxylating, and propargylating agents;
wherein said reactions are carried out within a single reaction vessel.

20. The process of claim 19, wherein the base is selected from the group consisting of sodium hydride, sodium methoxide, potassium methoxide, potassium carbonate, sodium carbonate, cesium carbonate, lithium carbonate, and ammonium carbonate.

21. The process of claim 19, wherein the base is selected from the group consisting of sodium hydride, sodium methoxide, and potassium carbonate.

22. The process of claim 19, wherein the adduct forming agent is selected from the group consisting of methyl iodide, methyl chloride, methyl bromide, 1-aminooxysulfonyl-2,4,6-trimethylbenzene, 0-(2,4-dinitrophenyl)hydroxylamine, hydroxylamine-O-sulfonic acid, allyl bromide, propargyl bromide, methoxymethyl bromide, benzyl chloride, and ethyl chloroacetate.

23. The process of claim 19, wherein steps of reacting the isocyanate C with the alkenoate D to form the unsubstituted pyrimidinedione A and reacting said unsubstituted pyrimidinedione A with the adduct forming agent are carried out in an organic solvent.

24. The process of claim 23, wherein the organic solvent is a polar aprotic solvent.

25. The process of claim 24, wherein the organic solvent is selected from the group consisting of N,N-dimethylformamide, dimethylacetamide, 1.3-dimethyl-3,4,5,6-tetrahydro-2(1H)-pyrimidinone, 1,3-dimethyl-2-imidazolidinone, N-methylpyrrolidinone, formamide, N-methylacetamide, N-methylformamide, acetonitrile, dimethyl sulfoxide, sulfolane, N,N-dimethylpropionamide, tetramethylurea, hexamethylphosphoramide

26. The process of claim 19, wherein the step of reacting the isocyanate C with the alkenoate D to form the unsubstituted pyrimidinedione A comprises heating the reaction mixture at about 70° C. to about 170° C. for about three to about 24 hours.

27. The process of claim 26, wherein the step of reacting the isocyanate C with the alkenoate D to form the unsubstituted pyrimidinedione A comprises heating the reaction mixture at about 100° C. to about 160° C. for about five to about 18 hours.

28. The process of claim 19, wherein the step of reacting the unsubstituted pyrimidinedione A with the adduct forming agent comprises reacting the unsubstituted pyrimidinedione A and adduct forming agent at about 0° C. to about 80° C. for at least 30 minutes.

29. The process of claim 28, wherein the step of reacting the unsubstituted pyrimidinedione A with the adduct forming agent comprises reacting the unsubstituted pyrimidinedione A and adduct forming agent at 0° C. to 40° C. for about one to about eight hours.

30. The process of claim 19, wherein:
about 0.01 to about 5 molar equivalents of isocyanate C are present per one molar equivalent of alkenoate D;
about 0.5 to about 10 molar equivalents of base are present per one molar equivalent of alkenoate D; and
about 0.01 to about 10 molar equivalents of adduct forming agent are present per one molar equivalent of unsubstituted pyrimidinedione A.

31. The process of claim 30, wherein:
about 0.5 to about 3 molar equivalents of isocyanate C are present per one molar equivalent of alkenoate D;
about 1 to about 5 molar equivalents of base are present per one molar equivalent of alkenoate D; and
about 1 to about 4 molar equivalents of adduct forming agent are present per one molar equivalent of unsubstituted pyrimidinedione A.
